(19) United States
(12) Patent Application Publication
(10) Pub. No.: US 2021/0251165 A1
(43) Pub. Date: Aug. 19, 2021

(54) TRIPLOID CANNABIS PLANTS AND METHODS FOR GENERATING SAME

(71) Applicant: CANOPY GROWTH CORPORATION, Smiths Falls (CA)

(72) Inventors: Kendra BEGLEY, Smiths Falls (CA); Ekaterina A. BOUDKO, Smiths Falls (CA); Jessica L. PARSONS, Smiths Falls (CA)

(21) Appl. No.: 17/255,709

(22) PCT Filed: Jul. 31, 2020
(86) PCT No.: PCT/CA2020/051055

§ 371 Date: Dec. 23, 2020

Related U.S. Application Data
(60) Provisional application No. 62/881,510, filed on Aug. 1, 2019.
(60) Provisional application No. 62/987,044, filed on Mar. 9, 2020.

(51) Int. Cl. A01H 1/00; A01H 1/08; A01H 6/28; A01H 5/12

(52) U.S. Cl. CPC A01H 1/101; A01H 5/12; A01H 6/28; A01H 1/08

(57) ABSTRACT

The present technology generally relates to a triploid Cannabis plant, triploid Cannabis seeds and to methods for generating such triploid Cannabis plant. The methods comprise crossing a tetraploid Cannabis plant with a diploid Cannabis plant to obtain F1 seeds, and growing a confirmed triploid F1 seed to generate the triploid Cannabis plant. In some embodiments, feminized pollen from a tetraploid Cannabis plant is crossed with a female diploid Cannabis plant.

### CROSS-REFERENCE TO RELATED APPLICATIONS

This application claims the benefit of and priority to U.S. provisional patent application No. 62/881,510, filed on Aug. 1, 2019; and U.S. provisional patent application No. 62/987,044, filed on Mar. 9, 2020; the content of both of which is herein incorporated in entirety by reference.

### FIELD OF TECHNOLOGY

The present technology generally relates to triploid Cannabis plants and seeds as well as to methods for generating same.

### BACKGROUND INFORMATION

Recently, there has been renewed interest in Cannabis due to its many medicinal effects, such as the treatment of epilepsy, pain, and nausea associated with cancer treatment (Andre et al., 2016; Thomas and Elsohly, 2016). While there are hundreds of different active metabolites present in Cannabis, two cannabinoids are present in high concentrations, and are generally considered to be the most important: Δ⁹-tetrahydrocannabinol (THC) and cannabidiol (CBD). THC is responsible for the well-known psychoactive properties of Cannabis whereas non-intoxicating CBD is widely used for pain, anxiety, depression, and sleep disorders (Andre et al., 2016; Corroon and Phillips, 2018). Another group of important chemicals is the terpenes, which contribute to the aroma and flavour of Cannabis products and also function as active metabolites (Russo, 2011; Andre et al., 2016). All of these metabolites are produced and stored within glandular trichomes that mainly develop on the inflorescence of the plant (Marks et al., 2009; Andre et al., 2016).

Several medicinal cannabinoid preparations are available. However, using whole Cannabis can be more effective than the single ingredient preparations for some conditions due to the synergy between multiple phytochemicals. In particular, CBD and the terpenes can modulate the effects of THC (Wilkinson et al., 2003; Brenneisen, 2007; Russo, 2011; Andre et al., 2016). Therefore, developing a wider variety of Cannabis strains may be preferable to new formulations of the active ingredients. Historically, new Cannabis strains have been developed through conventional breeding methods. However, these methods can be imprecise, and require several generations before the desired traits are obtained and a stable strain is produced.

One strategy to accelerate breeding development is a chromosome doubling event called polyploidization (Sattler et al., 2016). Polyploidization is common in the plant kingdom and has been associated with increased genetic diversity in some plant lineages (Comai, 2005). Desirable consequences of polyploidy for plant breeding include the buffering of deleterious mutations, increased heterozygosity, and hybrid vigor (Sattler et al., 2016). Consequently, polyploids often have phenotypic traits that are distinct from diploids, including larger flowers or leaves (Dermen, 1940; Rego et al., 2011; Trojak-Goluch and Skomra, 2013; Sattler et al., 2016; Talebi et al., 2017). Increases in active metabolite concentration in tetraploids are reported for numerous medicinal plants including Artemisia annua (Wallaart et al., 1999), Papaver somniferum (Mishra et al., 2010), Datura stramonium (Berkov and Philipov, 2002), Thymus persicus (Tavan et al., 2015), Echinacea purpurea (Abdoli et al., 2013) and Tanacetum parthenium (Majdi et al., 2010). Chemical mitotic inhibitory agents such as colchicine or dinitroanilines are often used to induce polyploidy in crop plants. A typical example is the production of tetraploid watermelon plants for the production of seedless triploid watermelon (Compton et al., 1996).

Cannabis is a diploid plant with 20 chromosomes (van Bakel et al., 2011). Doubling the chromosome set should allow more flexibility to increase potency or tailor the cannabinoid ratios. A handful of studies support the theory that polyploid Cannabis might have higher potency, although the results are mixed, with some studies finding decreases in THC (Clarke, 1981; Bagheri and Mansouri, 2015; Mansouri and Bagheri, 2017). However, these studies were conducted with hemp. Recently, studies on polyploid drug-type Cannabis strains demonstrated that tetraploidization did not significantly affect the chemical profile or overall plant growth or yield, although small increases in CBD and sesquiterpenes were observed (Parsons et al., 2019).

Triploids are plants with three sets of chromosomes, created when a diploid parent is crossed with a tetraploid parent. In general, when tetraploid plants are cross-fertilized with normal diploid plants, the seeds produced are triploid seeds. Triploid seeds produce triploid plants. The presence of an uneven number of chromosome sets in the triploid plants (three homologous sets of chromosome per somatic cell rather than the usual two) means that such plants often have poor reproductive viability, since the chromosomes may not be evenly segregated at meiosis to form viable gametes (pollen and egg cells). Triploid plants are thus often seedless and sterile. However, viability of triploid offspring varies between species. There are some cases where triploid plants can produce viable offspring, for example, arabidopsis and spinach (Comai et al. 2005).

Extensive research has been carried out on triploid hops. Hops is one of the most closely related species to Cannabis. Triploid hops plants are found to be more vigorous than either diploids or tetraploids and can exhibit unique phytochemical profiles. Triploid hop cultivation is particularly prevalent in Australia and New Zealand, where triploids are the main focus of hops breeding programs. So far, 12 cultivars have been released and seedless triploid hop cultivars make up 99% of cultivation (Beatson et al. 2003). Triploid hops are faster growing, higher yielding, and relatively seedless (Koutoulis et al. 2005). Some of these cultivars also display unique phytochemistry which results in alternative beer flavours (Beatson et al. 2003b). However, there have been no documented studies of ploid Cannabis plants.

For Cannabis, the presence of seeds in the bud product is a significant quality issue. To avoid seed production, lots must be visually inspected to identify and remove male plants and spontaneous male flowers. This process is time consuming and labour intensive and inefficient removal of pollen sources leads to seeds in the bud product, which greatly diminishes quality. The creation of sterile varieties should reduce the need for this visual inspection and allow for larger lots to be planted, while increasing the quality of the bud product. This should also allow for large scale outdoor grow operations without concern for impact on neighbouring farms or unintended pollination.

As such, there remains a need in the field of technology for methods and techniques for producing triploid Cannabis plants that alleviate at least some of the drawbacks of available cultivars.

### SUMMARY OF DISCLOSURE

Without wishing to be bound to any specific theory, embodiments of the present technology have been developed based on the advancements by the present investigators of techniques for inducing triploidy in Cannabis plant, in particular in Cannabis sativa. The present investigators have comprehended that a triploid Cannabis plant can be generated by crossing a tetraploid Cannabis plant (tetraploid parent) with a diploid Cannabis plant (diploid parent). As such and broadly speaking, embodiments of the present technology contemplate crossing a tetraploid Cannabis plant with a diploid Cannabis plant to obtain F1 seeds, and either collecting the triploid F1 seeds to obtain triploid seeds, or growing a confirmed triploid F1 seeds to generate the triploid Cannabis plant. Methods may be used for any species or strain of Cannabis and in particular for Cannabis sativa. Triploid plants and triploid seeds generated using the methods of the present technology are also provided. In some embodiments, triploid Cannabis plants of the present technology are sterile, i.e., they will not produce seeds, even in the presence of pollen.

In some embodiments of the present technology, a female tetraploid Cannabis plant is crossed with a male diploid Cannabis plant. In some embodiments, the female tetraploid Cannabis plant is grown from feminized seed (feminized seed is produced by inducing male flower formation on a female plant by, for example, using silver thiosulfate, as discussed further below; all plants germinated from feminized seed will be female). In some embodiments, the tetraploid Cannabis plant is a male plant and the diploid Cannabis plant is a female plant. In some embodiments, the diploid Cannabis plant is a female plant and is crossed with feminized pollen (i.e., pollen produced by inducing male flower formation on a female plant, for example using silver thiosulfate) from the tetraploid Cannabis plant.

In some embodiments of the present technology, the tetraploid Cannabis plant is obtained by treatment with an amount of a dinitroaniline compound sufficient to induce tetraploidy, such as oryzalin, effective to induce tetraploidy. In some embodiments of the present technology, the tetraploid Cannabis plant obtained by contacting a somatic tissue of the Cannabis plant to an amount of oryzaline sufficient to induce tetraploidy.

In some embodiments, the present technology relates to a method for generating a triploid Cannabis seed, the method comprising: obtaining a tetraploid Cannabis plant by contacting a somatic tissue of a Cannabis plant with an amount of a dinitroaniline compound; and crossing the tetraploid Cannabis plant with a diploid Cannabis plant to obtain a triploid F1 seed.

In some embodiments, the present technology relates to a method generating a triploid Cannabis plant, the method comprising crossing a tetraploid Cannabis plant parent with a diploid Cannabis plant parent to obtain triploid F1 seed; and growing the triploid F1 seed to generate the triploid Cannabis plant; wherein the triploid Cannabis plant obtained shows a reduction in total cannabinoid content compared to total cannabinoid content of the tetraploid parent and total cannabinoid content of the diploid parent. In some implementations of these embodiments, the reduction in total cannabinoid content is between about 30% and about 40%, or between about 32% and about 38%, or between about 33% and about 36%.

In some further implementations, the triploid Cannabis plant obtained by the methods of the present technology shows a reduction in total terpene content compared to total terpene content of the tetraploid parent and total terpene content of the diploid parent. In some instances, the reduction in total terpene content is of at least about 25%, or at least about 30%, or at least about 35%. In some instances, the triploid Cannabis plant of the present technology is a paternal triploid Cannabis plant.

In some embodiments, the present technology relates to a method for generating a triploid Cannabis plant, the method comprising: crossing a tetraploid Cannabis plant parent with a diploid Cannabis plant parent to obtain triploid F1 seed and growing the triploid F1 seed to generate the triploid Cannabis plant; wherein the triploid Cannabis plant obtained is substantially free of i) cannabidiolic acid (CBDA); ii) canabidiol (CBD); or iii) both i) and ii). As used herein, the expression “substantially free of cannabinoid” means an absence of cannabinoid or the presence of trace amounts of cannabinoids.

In some embodiments, the present technology relates to a method for generating a triploid Cannabis plant, the method comprising: crossing a tetraploid Cannabis plant parent with a diploid Cannabis plant parent to obtain triploid F1 seed; and ii) growing the triploid F1 seed to generate the triploid Cannabis plant; wherein the triploid Cannabis plant obtained has a Δ⁹-tetrahydrocannabinol:cannabidiol (THC:CBD) ratio that is greater than 150:1, or has a THC:CBD ratio that is between about 150:1 and about 300:1, or has a THC:CBD ratio that is between about 200:1 and about 250:1.

In some embodiments, the present technology also relates to triploid Cannabis plants and to triploid Cannabis seeds. In some instances, the triploid Cannabis plant of the present technology demonstrates a reduction in total cannabinoid content compared to its diploid parent and/or its tetraploid parent.

In some embodiments, the present technology also relates to a triploid Cannabis plant comprising a Cannabidivarinic acid (CBDVA) content of between about 0.05% and about 0.1% w/w, or between about 0.01% and about 0.07% w/w, or between about 0.02% and about 0.05% w/w.

In some embodiments, the present technology also relates to a triploid Cannabis plant comprising a Cannabichromenic acid (CBCA) content of between about 0.05% and about 1.0% w/w, or between about 0.05% and about 0.5% w/w, or between about 0.1% and about 0.5% w/w.

In some embodiments, the present technology also relates to a triploid Cannabis plant comprising a Tetrahydrocannabivarin (THCV) content of between about 0.05% and about 0.3% w/w, or between about 0.05% and about 0.2% w/w, or between about 0.1% and about 0.2% w/w.

In some embodiments, the present technology also relates to a triploid Cannabis plant comprising a Δ⁸-tetrahydrocannabinol (Δ⁸-THC) content of between about 0.001% and about 0.01% w/w; or between about 0.002% and about 0.005% w/w, or between about 0.003% and about 0.005% w/w.

In some embodiments, the present technology also relates to a triploid Cannabis plant comprising a Cannabigerolic acid (CBGA) content of between about 0.01% and about 0.5% w/w, or between about 0.05% and about 0.3% w/w, or between about 0.07% and about 0.3% w/w.

In some embodiments, the present technology also relates to a triploid Cannabis plant comprising a Cannabidiol (CBD) content of between about 0% and about 0.5% w/w, or between about 0% and about 0.3% w/w, or between about 0% and about 0.2% w/w.

In some embodiments, the present technology also relates to a triploid Cannabis plant comprising a Cannabidiol acid (CBDA) content of between about 0% and about 20% w/w, or between about 0% and about 15% w/w, or between about 0% and about 10% w/w.

In some embodiments, the present technology also relates to a triploid Cannabis plant comprising a Δ⁹-tetrahydrocannabinol (THC) content of between about 0.1% and about 1.0% w/w, or between about 0.1% and about 0.5% w/w, or between about 0.2% and about 0.5% w/w.

In some embodiments, the present technology also relates to a triploid Cannabis plant comprising a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w.

In some embodiments, the present technology also relates to a triploid Cannabis plant comprising one or more of: i) a Cannabidivarinic acid (CBDVA) content of between about 0.05% and about 0.1% w/w, or between about 0.01% and about 0.07% w/w, or between about 0.02% and about 0.05% w/w; ii) a Cannabichromenic acid (CBCA) content of between about 0.05% and about 1.0% w/w, or between about 0.05% and about 0.5% w/w, or between about 0.1% and about 0.5% w/w; iii) a Tetrahydrocannabivarin (THCV) content of between about 0.05% and about 0.3% w/w, or between about 0.05% and about 0.2% w/w, or between about 0.1% and about 0.2% w/w; iv) a Δ⁸-tetrahydrocannabinol (Δ⁸-THC) content of between about 0.001% and about 0.01% w/w; or between about 0.002% and about 0.005% w/w, or between about 0.003% and about 0.005% w/w; v) a Cannabigerolic acid (CBGA) content of between about 0.01% and about 0.5% w/w, or between about 0.05% and about 0.3% w/w, or between about 0.07% and about 0.3% w/w; vi) a Cannabidiol (CBD) content of between about 0% and about 0.5% w/w, or between about 0% and about 0.3% w/w, or between about 0% and about 0.2% w/w; vii) a Cannabidiolic acid (CBDA) content of between about 0% and about 20% w/w, or between about 0% and about 15% w/w, or between about 0% and about 10% w/w; viii) a Δ⁹-tetrahydrocannabinol (THC) content of between about 0.1% and about 1.0% w/w, or between about 0.1% and about 0.5% w/w, or between about 0.2% and about 0.5% w/w; and ix) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w.

In some embodiments, the present technology also relates to a triploid Cannabis plant comprising: i) a Δ⁹-tetrahydrocannabinol (THC) content of between about 0.1% and about 1.0% w/w, or between about 0.1% and about 0.5% w/w, or between about 0.2% and about 0.5% w/w; a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; or both; and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Δ⁹-tetrahydrocannabinol (THC) content of between about 0.1% and about 1.0% w/w, or between about 0.1% and about 0.5% w/w, or between about 0.2% and about 0.5% w/w; a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; or both; ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both; and iii) a Cannabigerolic acid (CBGA) content of between about 0.01% and about 0.5% w/w, or between about 0.05% and about 0.3% w/w, or between about 0.07% and about 0.3% w/w.

In some embodiments, the present technology also relates to a triploid Cannabis plant comprising: i) a Δ⁹-tetrahydrocannabinol (THC) content of between about 0.1% and about 1.0% w/w, or between about 0.1% and about 0.5% w/w, or between about 0.2% and about 0.5% w/w; a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; or both; ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both; and iii) a Cannabichromenic acid (CBCA) content of between about 0.05% and about 1.0% w/w, or between about 0.05% and about 0.5% w/w, or between about 0.1% and about 0.5% w/w.

In some embodiments, the present technology also relates to a triploid Cannabis plant comprising: i) a Δ⁹-tetrahydrocannabinol (THC) content of between about 0.1% and about 1.0% w/w, or between about 0.1% and about 0.5% w/w, or between about 0.2% and about 0.5% w/w; a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; or both; ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both; iii) a Cannabigerolic acid (CBGA) content of between about 0.01% and about 0.5% w/w, or between about 0.05% and about 0.3% w/w, or between about 0.07% and about 0.3% w/w; and iv) a Cannabichromenic acid (CBCA) content of between about 0.05% and about 1.0% w/w, or between about 0.05% and about 0.5% w/w, or between about 0.1% and about 0.5% w/w.

In some embodiments, the present technology also relates to a triploid Cannabis plant comprising: i) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; and ii) a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; ii) a Cannabidiolic acid (CBDA) content of between about 0% and about 20% w/w, or between about 0% and about 15% w/w, or between about 0% and about 10% w/w; or both; and iii) a Cannabigerolic acid (CBGA) content of between about 0.01% and about 0.5% w/w, or between about 0.05% and about 0.3% w/w, or between about 0.07% and about 0.3% w/w.

In some embodiments, the present technology also relates to a triploid Cannabis plant comprising: i) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; ii) a Cannabidiolic acid (CBDA) content of between about 0% and about 20% w/w, or between about 0% and about 15% w/w, or between about 0% and about 10% w/w; or both; and iii) a Cannabichromenic acid (CBCA) content of between about 0.05% and about 1.0% w/w, or between about 0.05% and about 0.5% w/w, or between about 0.1% and about 0.5% w/w.

In some embodiments, the present technology also relates to a triploid Cannabis plant comprising: i) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w; ii) a Cannabidiolic acid (CBDA) content of between about 0% and about 20% w/w, or between about 0% and about 15% w/w, or between about 0% and about 10% w/w; or both; iii) a Cannabigerolic acid (CBGA) content of between about 0.01% and about 0.5% w/w, or between about 0.05% and about 0.3% w/w, or between about 0.07% and about 0.3% w/w; and iv) a Cannabichromenic acid (CBCA) content of between about 0.05% and about 1.0% w/w, or between about 0.05% and about 0.5% w/w, or between about 0.1% and about 0.5% w/w.

In some embodiments, the present technology also relates to a triploid Cannabis plant comprising: i) a Cannabidivarinic acid (CBDVA) content of between about 0.05% and about 0.1% w/w, or between about 0.01% and about 0.07% w/w, or between about 0.02% and about 0.05% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Cannabichromenic acid (CBCA) content of between about 0.05% and about 1.0% w/w, or between about 0.05% and about 0.5% w/w, or between about 0.1% and about 0.5% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Tetrahydrocannabivarin (THCV) content of between about 0.05% and about 0.3% w/w, or between about 0.05% and about 0.2% w/w, or between about 0.1% and about 0.2% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Δ⁸-tetrahydrocannabinol (Δ⁸-THC) content of between about 0.001% and about 0.01% w/w; or between about 0.002% and about 0.005% w/w, or between about 0.003% and about 0.005% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Cannabigerolic acid (CBGA) content of between about 0.01% and about 0.5% w/w, or between about 0.05% and about 0.3% w/w, or between about 0.07% and about 0.3% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Δ⁹-tetrahydrocannabinol (THC) content of between about 0.1% and about 1.0% w/w, or between about 0.1% and about 0.5% w/w, or between about 0.2% and about 0.5% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising: i) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w and ii) a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology relates to a triploid Cannabis plant comprising: a Δ⁹-tetrahydrocannabinol (THC) content of between about 0.1% and about 1.0% w/w, or between about 0.1% and about 0.5% w/w, or between about 0.2% and about 0.5% w/w and a Cannabidivarinic acid (CBDVA) content of between about 0.05% and about 0.1% w/w, or between about 0.01% and about 0.07% w/w, or between about 0.02% and about 0.05% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology relates to a triploid Cannabis plant comprising: a Δ⁹-tetrahydrocannabinol (THC) content of between about 0.1% and about 1.0% w/w, or between about 0.1% and about 0.5% w/w, or between about 0.2% and about 0.5% w/w and a Cannabichromenic acid (CBCA) content of between about 0.05% and about 1.0% w/w, or between about 0.05% and about 0.5% w/w, or between about 0.1% and about 0.5% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology relates to a triploid Cannabis plant comprising: a Δ⁹-tetrahydrocannabinol (THC) content of between about 0.1% and about 1.0% w/w, or between about 0.1% and about 0.5% w/w, or between about 0.2% and about 0.5% w/w and a Cannabigerolic acid (CBGA) content of between about 0.01% and about 0.5% w/w, or between about 0.05% and about 0.3% w/w, or between about 0.07% and about 0.3% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology relates to a triploid Cannabis plant comprising: a Cannabidivarinic acid (CBDVA) content of between about 0.05% and about 0.1% w/w, or between about 0.01% and about 0.07% w/w, or between about 0.02% and about 0.05% w/w and a Cannabichromenic acid (CBCA) content of between about 0.05% and about 1.0% w/w, or between about 0.05% and about 0.5% w/w, or between about 0.1% and about 0.5% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology relates to a triploid Cannabis plant comprising: a Cannabichromenic acid (CBCA) content of between about 0.05% and about 1.0% w/w, or between about 0.05% and about 0.5% w/w, or between about 0.1% and about 0.5% w/w and a a Cannabigerolic acid (CBGA) content of between about 0.01% and about 0.5% w/w, or between about 0.05% and about 0.3% w/w, or between about 0.07% and about 0.3% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising one or more of: i) a Tetrahydrocannabivarin (THCV) content of between about 0.05% and about 0.3% w/w, or between about 0.05% and about 0.2% w/w, or between about 0.1% and about 0.2% w/w; ii) a Δ⁸-tetrahydrocannabinol (Δ⁸-THC) content of between about 0.001% and about 0.01% w/w; or between about 0.002% and about 0.005% w/w, or between about 0.003% and about 0.005% w/w; iii) a Δ⁹-tetrahydrocannabinol (THC) content of between about 0.1% and about 1.0% w/w, or between about 0.1% and about 0.5% w/w, or between about 0.2% and about 0.5% w/w; and iv) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising one or more of: i) a Δ⁸-tetrahydrocannabinol (Δ⁸-THC) content of between about 0.001% and about 0.01% w/w; or between about 0.002% and about 0.005% w/w, or between about 0.003% and about 0.005% w/w; ii) a Δ⁹-tetrahydrocannabinol (THC) content of between about 0.1% and about 1.0% w/w, or between about 0.1% and about 0.5% w/w, or between about 0.2% and about 0.5% w/w; and iii) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plants comprising one or more of: i) a Cannabidivarinic acid (CBDVA) content of between about 0.05% and about 0.1% w/w, or between about 0.01% and about 0.07% w/w, or between about 0.02% and about 0.05% w/w; ii) a Cannabichromenic acid (CBCA) content of between about 0.05% and about 1.0% w/w, or between about 0.05% and about 0.5% w/w, or between about 0.1% and about 0.5% w/w; iii) a Tetrahydrocannabivarin (THCV) content of between about 0.05% and about 0.3% w/w, or between about 0.1% and about 0.2% w/w; iv) a Cannabigerolic acid (CBGA) content of between about 0.01% and about 0.5% w/w, or between about 0.05% and about 0.3% w/w; and v) a Tetrahydrocannabivarin (THCV) content of between about 0.05% and about 0.3% w/w, or between about 0.05% and about 0.2% w/w, or between about 0.1% and about 0.2% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plants comprising one or more of: i) a Δ⁸-tetrahydrocannabinol (Δ⁸-THC) content of between about 0.001% and about 0.01% w/w; or between about 0.002% and about 0.005% w/w, or between about 0.003% and about 0.005% w/w; ii) a Δ⁹-tetrahydrocannabinol (THC) content of between about 0.1% and about 1.0% w/w, or between about 0.1% and about 0.5% w/w, or between about 0.2% and about 0.5% w/w; and iii) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plants comprising one or more of: In some embodiments, the present technology also relates to a triploid Cannabis plant comprising one or more of: i) a Cannabichromenic acid (CBCA) content of between about 0.05% and about 1.0% w/w, or between about 0.05% and about 0.5% w/w, or between about 0.1% and about 0.5% w/w; ii) a Cannabigerolic acid (CBGA) content of between about 0.01% and about 0.5% w/w, or between about 0.05% and about 0.3% w/w, or between about 0.07% and about 0.3% w/w; and iii) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising one or more of i) a Cannabigerolic acid (CBGA) content of between about 0.01% and about 0.5% w/w, or between about 0.05% and about 0.3% w/w, or between about 0.07% and about 0.3% w/w; and ii) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some embodiments, the present technology also relates to a triploid Cannabis plant comprising one or more of: i) a Cannabidivarinic acid (CBDVA) content of between about 0.05% and about 0.1% w/w, or between about 0.01% and about 0.07% w/w, or between about 0.02% and about 0.05% w/w; ii) a Cannabichromenic acid (CBCA) content of between about 0.05% and about 1.0% w/w, or between about 0.05% and about 0.5% w/w, or between about 0.1% and about 0.5% w/w; iii) a Tetrahydrocannabivarin (THCV) content of between about 0.05% and about 0.3% w/w, or between about 0.05% and about 0.2% w/w, or between about 0.1% and about 0.2% w/w; iv) a Δ⁸-tetrahydrocannabinol (Δ⁸-THC) content of between about 0.001% and about 0.01% w/w; or between about 0.002% and about 0.005% w/w, or between about 0.003% and about 0.005% w/w; v) a Cannabigerolic acid (CBGA) content of between about 0.01% and about 0.5% w/w, or between about 0.05% and about 0.3% w/w, or between about 0.07% and about 0.3% w/w; vi) a Δ⁹-tetrahydrocannabinol (THC) content of between about 0.1% and about 1.0% w/w, or between about 0.1% and about 0.5% w/w, or between about 0.2% and about 0.5% w/w; vii) a Δ⁹-tetrahydrocannabinolic acid (THCA) content of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some implementations of these embodiments, the triploid Cannabis plant further comprises a Cannabidiol (CBD) content of between about 0% and about 0.5% w/w, or between about 0% and about 0.3% w/w, or between about 0% and about 0.2% w/w; or a Cannabidiolic acid (CBDA) content of between about 0% and about 20% w/w, or between about 0% and about 15% w/w, or between about 0% and about 10% w/w; or both.

In some implementations of these embodiments, the triploid Cannabis plant further comprises a α-Pinene content of between about 0.04% and about 0.1% w/w, or between about 0.5% and about 0.1% w/w.

In some implementations of these embodiments, the triploid Cannabis plant further comprises a β-Pinene content of between about 0.02% and about 0.5% w/w, or between about 0.3% and about 0.5% w/w.

In some implementations of these embodiments, the triploid Cannabis plant further comprises a Myrcene content of between about 0.1% and about 0.5% w/w or between about % and about % w/w, or between about 0.2% and about 0.4% w/w.

In some implementations of these embodiments, the triploid Cannabis plant further comprises a Limomene content of between about 0.01% and about 0.05% w/w, or between about 0.2% and about 0.4% w/w.

In some implementations of these embodiments, the triploid Cannabis plant further comprises an Ocimene content of between about 0% and about 0.005% w/w, or between about 0% and about 0.004% w/w.

In some implementations of these embodiments, the triploid Cannabis plant further comprises a Linalool content of between about 0.01% and about 0.03% w/w, or between about 0.1% and about 0.2% w/w.

In some implementations of these embodiments, the triploid Cannabis plant further comprises a γ-Terpineol content of between about 0.02% and about 0.05% w/w, or between about 0.03% and about 0.05% w/w.

In some implementations of these embodiments, the triploid Cannabis plant further comprises a Geraniol content of between about 0.001% and about 0.02% w/w, or between about 0.002% and about 0.01% w/w.

In some implementations of these embodiments, the triploid Cannabis plant further comprises a β-Caryophyllene content of between about 0.01% and about 0.05% w/w, or between about 0.02% and about 0.05% w/w.

In some further implementations of these embodiments, the triploid Cannabis plant, further comprises one or more of: i) a α-Pinene content of between about 0.04% and about 0.1% w/w, or between about 0.5% and about 0.1% w/w; ii) a β-Pinene content of between about 0.02% and about 0.5% w/w, or between about 0.3% and about 0.5% w/w; iii) a Myrcene content of between about 0.1% and about 0.5% w/w or between about % and about % w/w, or between about 0.2% and about 0.4% w/w; iv) a Limomene content of between about 0.01% and about 0.05% w/w, or between about 0.2% and about 0.4% w/w; v) a Linalool content of between about 0.01% and about 0.03% w/w, or between about 0.1% and about 0.2% w/w; and vi) a β-Caryophyllene content of between about 0.01% and about 0.05% w/w, or between about 0.02% and about 0.05% w/w.

In some further implementations of these embodiments, the triploid Cannabis plant further comprises one or more of: i) a α-Pinene content of between about 0.04% and about 0.1% w/w, or between about 0.5% and about 0.1% w/w; ii) a Myrcene content of between about 0.1% and about 0.5% w/w, or between about 0.2% and about 0.4% w/w; and iii) a β-Caryophyllene content of between about 0.01% and about 0.05% w/w, or between about 0.02% and about 0.05% w/w.

In some implementations of these embodiments, the triploid Cannabis plant further comprises one or more of: i) a α-Pinene content of between about 0.04% and about 0.1% w/w, or between about 0.5% and about 0.1% w/w; ii) a β-Pinene content of between about 0.02% and about 0.5% w/w, or between about 0.3% and about 0.5% w/w; iii) a Myrcene content of between about 0.1% and about 0.5% w/w or between about % and about % w/w, or between about 0.2% and about 0.4% w/w; iv) a Limomene content of between about 0.01% and about 0.05% w/w, or between about 0.2% and about 0.4% w/w; v) an Ocimene content of between about 0% and about 0.005% w/w, or between about 0% and about 0.004% w/w; vi) a Linalool content of between about 0.01% and about 0.03% w/w, or between about 0.1% and about 0.2% w/w; vii) a γ-Terpineol content of between about 0.02% and about 0.05% w/w, or between about 0.03% and about 0.05% w/w; viii) comprises a Geraniol content of between about 0.001% and about 0.02% w/w, or between about 0.002% and about 0.01% w/w; and ix) a β-Caryophyllene content of between about 0.01% and about 0.05% w/w, or between about 0.02% and about 0.05% w/w.

In some embodiments, the triploid Cannabis plant of the present technology comprises a total terpene content of between about 0.1% and about 2.0% w/w, or between about 0.1% and about 1.0% w/w, or between about 0.3% and about 0.8% w/w.

In some embodiments, the triploid Cannabis plant of the present technology shows an increase in Δ⁹-tetrahydrocannabinolic acid (THCA) content compared to a tetraploid Cannabis plant and/or a diploid Cannabis strand. In some implementations of these embodiments, the increase in THCA content is of at least 25%, or of at least 30%, or of at least 35%, or of at least 40%.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of how endopolyploidy occurs naturally in some of the cells of a plant; and

FIG. 2 is a flow diagram of a method for producing a parental tetraploid plant of Cannabis sativa according to one embodiment of the present technology.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “comprise” is used in its non-limiting sense to mean that items following the word “comprise” are included, but items not specifically mentioned are not excluded.

As used herein, the term “strain” can be used interchangeably with “genotype” and refers to the DNA sequence of the genetic makeup of a cell, and therefore of a plant, which determines a specific characteristic (phenotype) of that plant. As used herein the term refers to different variants of a species of plant and is used interchangeably. Examples of strains or genotypes of Cannabis include, but are not limited to: Hindu Kush®, Skunk Haze®, Super Nordle®, Cannatonic®, Sour Kush®, Acapulco Gold®, Wonder Diesel®, and Black Gold®. As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species. For example some describe Cannabis plants as a single species, C. sativa L., with multiple subspecies while others classify Cannabis plants into multiple species, most commonly as C. sativa L. and C. indica Lam. and sometimes additionally as C. ruderalis Janisch., depending on multiple criteria including morphology, geographic origin, chemical content, and genetic measurements. Regardless, all plants of genus Cannabis can interbreed and produce fertile offspring. As used herein, the expression “Cannabis plant” includes hemp plant and as such, the expression “triploid Cannabis plant” includes triploid hemp plant.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids. In another embodiment, a cannabinoid is one of a class of diverse chemical compounds that may act on cannabinoid receptors such as CB₁ and CB₂ in cells that alter neurotransmitter release in the brain. In many cases, a cannabinoid can be identified because its chemical name will include the text string “*cannabi*”. However, there are a number of cannabinoids that do not use this nomenclature, such as for example those described herein.

As used herein, the expression “% w/w” or “% by weight” is calculated based on dry weight of the total material.

As used herein, the term “diploid” refers to organisms or cells with two complete chromosome sets (2n), typically in the somatic cells of a plant. A Cannabis sativa diploid (2n) plant with a complete set of chromosomes has 20 chromosomes. As used herein, the term “polyploid” refers to a plant having more than the usual number (two) of chromosome sets, including three or more chromosome sets, four or more, five or more, etc. A true polyploid will have the extra chromosome sets in all cells, but ploidy can vary between tissues and is sometimes not passed on to the seeds. Polyploid can refer to organisms with three or more complete chromosome sets in all somatic cells. Polyploid can refer to organisms with three or more complete chromosome sets in one or more tissues. Polyploids can include, but are not limited to, triploids (3n), tetraploids (4n), hexaploids (6n), and octaploids (8n). As used herein, the expression “stable polyploid plant” refers to a plant that retains its polyploid number in some, most, or all tissues for a few months and/or through multiple generations. For example, the expression “stable tetraploid plant” refers to a plant that retains its tetraploid number in some, most, or all tissues for a few months and/or through multiple generations.

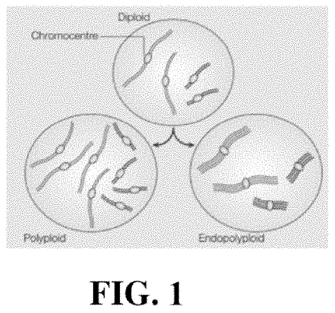

The term “endopolyploidy” as used herein refers to a natural doubling of the DNA content brought about by the process of endoreduplication in the plant cell which occurs when the cell undergoes a DNA replication without cell division, however, the number of chromosomes remains the same (FIG. 1). The term “aneuploid” as used herein refers to the situation when particular chromosomes are under or over-represented, but the entire chromosome set is not multiplied. For example, an aneuploid cell or plant may have 3 copies of chromosome numbers 1, 5, and 6, but only two copies of the other chromosomes.

As used herein, the term “mixoploid” plant refers to a plant having a mix of different ploidy cells within one tissue of the plant. For example, both diploid and tetraploid cells are present in the leaves. In some cases, the tissue or tissues are composed of some polyploid and some diploid cells.

As used herein, the expression “root tip squash” refers to a method whereby actively dividing cells from the root tips of a plant are isolated, stained, and mounted on a slide so that the chromosomes may be observed and/or counted under a microscope.

As used herein, the term “chlorosis” refers to the condition where leaves lose their green pigmentation, which can be caused by nutrient deficiency, lack of light, or disease.

Polyploidization is a powerful tool for improving desirable plant characteristics and is an effective method to induce variation. The method of polyploidization can result in a plant that has increased value for medicinal uses and a plant that is stable enough to use in the medical industry. Triploidization can result in a plant that is seedless and/or sterile. Because of the allogamous nature of the fertilization of the species (the fertilization of a flower by pollen from another flower, especially one on a different plant), it is difficult to maintain the plant's potency and efficacy if grown from the seeds. Therefore, tissue culture is often the most suitable way to maintain their genetic lines (although some of the plants used for medicinals are monoecious). In some cases, however, triploid hybrids may be created by crossing two true breeding parental lines (diploid and tetraploid).

In one embodiment, the present technology relates to a method of generating a triploid Cannabis plant. In some implementations of this embodiment, the present technology relates to a method of generating a triploid Cannabis plant seed. In some embodiments, the present technology relates to triploid Cannabis plants, seeds and cells, made using the methods provided herein.

Generation of triploidy in Cannabis plants is obtained in the present technology by crossing a tetraploid Cannabis plant (also referred to herein as a tetraploid parent) with a diploid Cannabis plant (also referred to herein as a diploid parent). F1 seeds are collected; and a confirmed triploid F1 seed is then grown to generate the triploid Cannabis plant. Triploidy of the F1 seeds is generally confirmed by germinating the seeds and confirming triploidy of the seedlings, although any standard technique may be used.

The parental tetraploid Cannabis plant for use in the methods of the present technology may be obtained using techniques known in the art or as described below, and is not meant to be particularly limited. In some embodiments, the parental tetraploid Cannabis plant is female (and the parental diploid Cannabis plant is male). In some embodiments where the parental tetraploid Cannabis plant is female, the parental tetraploid Cannabis plant is grown from feminized seed. In alternative embodiments, the parental tetraploid Cannabis plant is male (and the parental diploid Cannabis plant is female). In some such embodiments, feminized pollen produced by a female tetraploid Cannabis plant that is induced to form male flowers (as discussed further below) is crossed with a female parental diploid Cannabis plant.

As used herein, the term “feminized” refers to seeds produced in a way to make sure that all the resulting plants grown from the seeds are female. This is highly desirable since only female Cannabis plants make buds. Also, fertilization of female plants by the male plants leads to seed production, which is detrimental to crop quality. Use of feminized seeds can mean that all plants end up producing buds; there is less wasted space from growing (male) plants that need to be thrown away; there is no need to watch plants closely to identify and remove the male plants or spontaneous male flowers; and there is reduced pollination so that seeds are not formed. In contrast to feminized seeds, regular or non-feminized seeds generally produce about 40-50% male plants. Feminized seeds are generally produced by inducing male flower formation on a female plant, thereby producing pollen with only female chromosomes (also referred to herein as “feminized” pollen).

Breeders generally create feminized seeds by breeding two female cannabis plants together, which means that all resulting offspring will be female. Generally, this is done by inducing male flower formation on a female plant, thereby producing pollen with only female chromosomes (feminized pollen). Male flower formation is often induced by spraying developing flowers with a substance that changes flower development to force one of the female plants to start producing pollen sacs like a male plant. The feminized pollen is then used to pollinate buds of a different female plant.

Parental plants (tetraploid and/or diploid) used in the present technology may be induced to form male flowers using any suitable method. Non-limiting examples of substances that can be used to chemically induce male flower formation on a female plant (and thereby produce “feminized” pollen and seeds) include colloidal silver, silver thiosulfate (STS), silver ion, silver nitrate, gibberellic acid, cobalt ion, cobalt(II) chloride, and other ethylene inhibitors. It should be understood that the method or chemical used to induce male flowers on female Cannabis plants is not particularly limited. Any standard technique may be used, including but not limited to methods described in Ram and Sett, 1979; Ram and Jaiswal, 1972; Sarath and Ram, 1978; and Ram and Sett, 1982. Further, length and strength of such treatment (e.g., STS concentration) will be selected or adjusted by the skilled artisan based on various characteristics such as the genotype of the plant, growing conditions, etc.

In one embodiment, male flowers are induced on female plants by spraying the nodes of a plant with a 3 mM solution of silver thiosulphate for 12 consecutive days in the first two weeks of flowering. This treatment causes the female plants to produce male flowers and pollen (all of the pollen being “feminized”, i.e., containing female gametes, resulting in all-female seeds). Following an appropriate amount of time, mature seeds may be collected from these crosses and germinated. The seedlings can be tested using a ploidy analyzer to confirm that they are triploid.

Plants can also be feminized by growing them in stress conditions, which leads to production of mixed-gender plants which are self-fertilizing hermaphrodites (also referred to as “hermies”), however these are less desirable as hermies are detrimental to crop quality and will produce more male plants than chemically feminized seeds.

In one embodiment, the tetraploid Cannabis plant used in the methods of the present technology is a female tetraploid Cannabis plant grown from feminized tetraploid seeds. In another embodiment, feminized tetraploid pollen is used (i.e., pollen from a male flower induced on a tetraploid female plant is used, and crossed with a female diploid plant). In some embodiments, seeds are feminized (male flowers are induced) using STS.

In another embodiment, the diploid Cannabis plant used in the methods of the present technology is a female diploid Cannabis plant grown from feminized diploid seeds. In another embodiment, feminized diploid pollen is used (i.e., pollen from a male flower induced on a diploid female plant is used, and crossed with a female tetraploid plant, which may or may not itself be grown from feminized seeds).

In some embodiments, both the parental tetraploid and the parental diploid Cannabis plant used in the methods of the present technology are grown from feminized seeds.

Tetraploidy in parental plants used in the methods of the present technology may be induced using any suitable technique. Non-limiting examples of methods used to induce tetraploidy include: treating a Cannabis plant with an amount of a dinitroaniline compound, such as benfluralin, butralin, chlornidine, dinitramine, dipropalin, ethalfluralin, fluchloralin, isopropalin, methalpropalin, nitralin, oryzalin, pendimethalin, prodiamine, profluralin, and trifluralin or derivatives thereof; treating a plant with colchicine; or treating a plant with another suitable chemical mitotic inhibitory agent. Typically the plant or a portion thereof is contacted with the compound. For example, somatic plant tissue such as an axillary bud of the Cannabis plant is treated with the compound; allowed to grow in tissue culture; and then planted in soil. Such methods have been described in International Patent Application No. PCT/CA2019/050332, filed Mar. 19, 2019, and in Parsons et al., 2019, the entire content of each of which is hereby incorporated by reference.

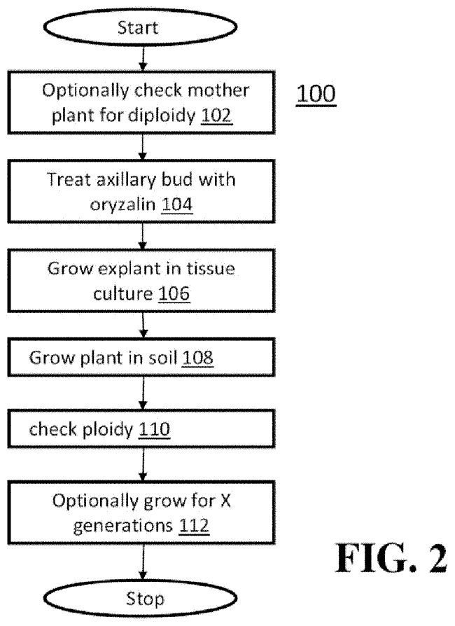

An example of one such method 100 for inducing tetraploidy in Cannabis sativa according to one embodiment of the present technology is outlined in FIG. 2, wherein oryzalin is used as the dinitroaniline compound. In this embodiment, the mother plants may first be optionally confirmed for diploidy (step 102) using methods known to those of skill in the art such as the root tip squash method and/or by flow cytometry. Somatic tissue such as for example, the axillary buds are then treated with oryzalin in an amount and for a time sufficient to induce tetraploidy (step 104). For instance, the axillary buds may be soaked in a solution of oryzalin at a concentration effective to induce tetraploidy (step 104). The axillary buds of C. sativa can be excised using any method known in the art. The effective concentration of oryzalin may vary depending on the strain or genotype of C. sativa used. The method may include soaking the excised somatic tissue (e.g., axillary buds) of C. sativa in a concentration of oryzalin strong enough to induce polyploidy or more specifically, tetraploidy but not so strong that it induces toxicity in the axillary buds resulting in death of a high percentage of the resulting plants. The excised axillary buds of C. sativa are soaked in the composition of oryzalin for a time sufficient to induce polyploidy in the treated somatic tissue. The time needed to induce polyploidy may vary depending on the strain or genotype being used. The treated somatic tissue is then transplanted and grown in tissue culture producing a plantlet (step 106) and is grown in tissue culture until it roots. The method of growth in tissue culture can include growth in a semi-solid media containing shoot elongation hormones. In some embodiments, the explant is kept in shoot elongation medium until it is ready to be transferred to medium containing a rooting hormone. In some embodiments, the explants are left in the shoot elongation medium until shoots form. The resulting plantlet is then transferred to soil, also called acclimatization or “hardening off” (step 108). The media from the tissue culture (including a gelling agent) can be gently broken up (with forceps or using any other method that does not result in damage to the roots) and the plantlet can be removed from the culture container.

In some embodiments, the plants are covered with a humidity dome and vented periodically to reduce the amount of humidity that builds up in the dome. In some embodiments, the plants are covered with a humidity dome for between about 1 and about 5 weeks, including but not limited to between about 1 week, about 1.5 weeks, about 2 weeks, about 2.5 weeks, about 3 weeks, about 3.5 weeks, about 4 weeks and about 4.5 weeks. In some embodiments, after between about 1 week and about 5 weeks in the humidity dome, the dome is removed and the plants are allowed to grow in a typical environment and the plant health is assessed with time.

In step 110, the plant, an explant, or a plantlet is tested for tetraploidy using any methods know in the art. In some embodiments, one or more plant tissues is initially tested via flow cytometry and then the results are confirmed using the root tip squash method where the chromosomes in the root tip are imaged and counted. In some embodiments tetraploidy is checked in the young leaves via flow cytometry. In some embodiments tetraploidy is checked in both the young and older leaves via flow cytometry. In some embodiments, tetraploidy is checked in the root tips via the root tip squash method. In some embodiments the plant is tested for mixoploidy.

The plant may be grown until it is flowering and tested for cannabinoids and other substances (step 112). At this point the plant may be allowed to grow for a few months to make sure it is a stable polyploid, i.e., a stable tetraploid. The plant may be allowed to grow for multiple generations to identify that it is a stable tetraploid.

In some other embodiments, method 100 may not have all of the above steps and/or may have other steps in addition to or instead of those listed above. The steps of method 100 may be performed in another order. Subsets of the steps listed above as part of method 100 may be used to form their own method.

According to some embodiments, the expression in “an amount sufficient to induce polyploidy (or tetraploidy)” refers to a concentration of dinitroaniline compound which is effective to induce polyploidy or tetraploidy in a Cannabis plant. In some implementations, the effective amount is between about 5 μM and about 200 μM, or between about 10 μM and about 200 μM, or between about 50 μM and about 200 μM, about 5 μM and about 100 μM, or between about 10 μM and about 100 μM, or between about 20 μM and about 150 μM, or between about 20 μM and about 60 μM, or between about 50 μM and about 150 μM, or between about 50 μM and about 100 μM; or is about 5 μM, or about 10 μM, or about 15 μM, or about 20 μM, or about 25 μM, or about 30 μM, or about 35 μM, or about 40 μM, or about 45 μM, or about 50 μM, or about 55 μM, or about 60 μM, or about 65 μM, or about 70 μM, or about 75 μM, or about 80 μM, or about 85 μM, or about 90 μM, or about 95 μM, or about 100 μM, or about 105 μM, or about 110 μM, or about 115 μM, or about 120 μM, or about 125 μM, or about 130 μM, or about 135 μM, or about 140 μM, or about 145 μM.

In some embodiments, the method of preparation of the dinitroaniline compound includes dissolving the dinitroaniline compound in a suitable solvent and then diluting the resulting composition with media to obtain a suitable or desired concentration of the dinitroaniline compound. In some embodiments, the media comprises sucrose and salts (for example, the media may comprise 30 g/L sucrose and 4.43 g/L MS basal salts). In such embodiments wherein the dinitroaniline compound is oryzalin, ethanol (e.g., 80%) is used because oryzalin is not soluble in water. The ethanol may also serve to disrupt cell membrane of the axillary bud to allow contact between the oryzalin and the chromosomes. The concentration of ethanol in the resulting oryzalin mixture may be less than about 0.5%, including but not limited to less than about 0.45%, about 0.4%, about 0.35%, about 0.3%, about 0.25%, about 0.2%, about 0.15%, about 0.1%, or about 0.5%. In some instances, the concentration of ethanol is less than about 0.25%. Optionally, before transplantation, the axillary bud is rinsed with sterile water with about 1 ml/L PPM™ (a plant preservative mixture). In other instances, the concentration of PPM™ is between about 0.5 and about 2 ml/L PPM™ including about 0.6, about 0.7, about 0.8, about 0.9, about 1.0, about 1.1, about 1.2, about 1.3, about 1.4, about 1.5, about 1.6, about 1.7, about 1.8, or about 1.9 ml/L PPM™.

According to some embodiments, the expression in “a time sufficient to induce polyploidy (or tetraploidy)” refers to a period of time during which the Cannabis plant is treated with the dinitroaniline compound that is sufficient to induce polyploidy or tetraploidy in the Cannabis plant. In some implementations, the time sufficient to induce polyploidy is between about 24 hours and about 48 hours, or between about 12 hours and about 48 hours, or is about 13 hours, about 14 hours, about 15 hours, about 16 hours, about 17 hours, about 18 hours, hours 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, hours 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, shoot elongation medium is 4.44 g/L Murashige & Skoog (MS) basal media with vitamins. In some embodiments, the shoot elongation medium includes naphthaleneacetic acid (NAA). In some embodiments, the shoot elongation medium includes kinetin (KIN). In some embodiments, the NAA is included at a concentration of between about 0.05 to about 0.5 mg/L, including about 0.1 mg/L to about 0.5 mg/L, including but not limited to about, 0.2, 0.3, and 0.4 mg/L. In some embodiments, the kinetin is used at a concentration of about 0.2 to about 2.0 mg/L. In some embodiments, the KIN is included at a concentration of about 0.4 mg/L to about 1 mg/L, including but not limited to about, 0.5, 0.6, 0.7, 0.8, and 0.9 mg/L.

In some embodiments, the explant is kept in the shoot elongation medium until it is ready to be transferred to medium containing a rooting hormone. In some embodiments, the explants are left in the shoot elongation medium until shoots form. In some embodiments, the explant (treated axillary bud) is grown in a semi-solid media containing a gelling agent such as Gelzan™ or Agar. In some embodiments the Gelzan™ is included at a concentration of about 4 mg/L. In some embodiments, charcoal is added to the semi-solid media at a concentration of from about 0.1 mg/L to about lmg/L, including 0.5 mg/L. In some embodiments, the amount of time the explants are left in the shoot elongation medium is between about 1 week and about 16 weeks, or is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks total. In some embodiments, the time required for rooting and obtaining shoots is between about 4 weeks and about 8 weeks. In some embodiments, the explants start rooting in the elongation media and do not require rooting media.

In some embodiments, the explant is transferred to a media containing a rooting hormone. In some embodiments, the rooting hormone comprises indole-3-butyric acid (IBA). In some instances, the IBA is used at a concentration of between about 0.5 mg/L and about 2 mg/L, or at a concentration of about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, or about 1.9 mg/L. In some instances, the time required for rooting is between about 1 week and about 16 weeks, or is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks. In some embodiments, the time required for rooting is between about 4 weeks and about 8 weeks.

A widespread consequence of polyploidy is an increase in cell size, caused by a larger number of gene copies. However, an increase in cell size does not always translate to increased size of the whole plant or its organ, since the number of cell divisions in polyploids can be reduced (Sattler et al., 2016). In some embodiments of the present technology, the tetraploid plants have fan leaves and/or stomata that are larger than those of diploids. In some instances the tetraploids may also exhibit a lower density of stomata and stomata guard cells with larger length and diameter, but shorter and wider leaves, compared to diploids (Mansouri and Bagheri, 2017). Changes in stomata size and density are common among tetraploids (Ascough et al., 2008; Sakhanokho et al., 2009; Rego et al., 2011; Talebi et al., 2017). Overall, these data suggest that stomata size and density are reliable phenotypic markers for at least some polyploid Cannabis, particularly tetraploid and triploid Cannabis.

The major cannabinoids THC and CBD in acid form are produced from a common cannabigerolic acid precursor by THCA synthase and CBDA synthase, respectively (Andre et al., 2016). The cannabinoid ratio is determined by codominant alleles of these synthase enzymes, which occur at a single locus on chromosome 6 (de Meijer et al., 2003; Marks et al., 2009). A number of allelic variants of these enzymes exist in different cultivars, and each has a unique effect on cannabinoid production. Therefore, large-scale genome rearrangements or duplications such as polyploidization could enable new allelic combinations, which have the potential to create novel chemotypes (Laverty et al., 2018).

In some embodiments of the present technology, the cannabinoid profile of the triploid Cannabis plant is changed relative to diploids and/or relative to tetraploids. In some embodiments, THCA content, CBDA content, cannabigerol acid precursor content, THC content, and/or CBD content is changed in the triploid plant, compared to diploids and/or tetraploids. In alternative embodiments, the cannabinoid profile of the triploid plant (e.g., THCA content, CBDA content, cannabigerol acid precursor content, THC content, and/or CBD content) is unchanged relative to diploids and/or tetraploids. In some instances, ploidy may have limited influence on the cannabinoid biosynthetic pathway.

Terpenes are important aromatic compounds that determine the smell and taste of Cannabis products, and also modulate the drug effects of cannabinoids. Terpene concentrations above 0.5 mg g⁻¹ are considered pharmacologically relevant (Russo, 2011). In some embodiments of the present technology, the triploid plants have increased or decreased terpene content compared to diploids and/or tetraploids. For example, α-Pinene content, β-Pinene content, Myrcence content, Limomene content, Ocimene content, Linalool content, γ-Terpineol content, Geraniol content, β-Caryophyllene content, α-humulene content, α-bisabolol content, cis-nerolidol content, nerolidol content, sesquiterpenes, monoterpenes, and/or total terpene content may be increased or decreased in the triploid plants compared to diploids and/or tetraploids. In some embodiments of the present technology, total terpene content is increased in the triploid Cannabis plants. In some instances, terpene levels are increased in both leaves and buds. Trichome density on flowers is also increased in some embodiments. In some embodiments of the present technology, total terpene content is decreased in the triploid Cannabis plants. In some instances, terpene levels are decreased in both leaves and buds. Trichome density on flowers is also decreased in some embodiments.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Use of Polyploid Strains for the Breeding of Triploid Cannabis Cultivars

Initial experiments are carried out with the Super Nordle® (first set of experiments) and Cannatonic® (second set of experiments) strains. In order to cross female diploids and tetraploids, female plants are induced to create male flowers using a silver thiosulphate spray, Four diploid plants and four tetraploid plants are treated as a comparison in the initial experiments, to confirm that pollenIseeds can be produced from both types of plants. Tetraploid plants are obtained as described herein and/or as described in International Patent Application No. PCT/CA2019/050332, filed Mar. 19, 2019. Tetraploids with induced male flowers are placed with six diploid females and vice versa, so that all seeds produced should be triploid. Two plants of the same ploidy are placed with the pollen donor plants as a control to compare seed set. Seeds are harvested from individual plants so that seed generation per plant can be calculated with 8 replicates (see overview below). The generation of male flowers and pollen viability are assessed and compared in pollen donor diploid and tetraploid plants in two ways. Pollen from each plant is collected and stained using a 2,3,5-triphenyl tetrazolium chloride stain and a sucrose media to monitor pollen tube growth. Pollen viability tests are conducted 3 and 5 weeks after the initiation of male flower induction treatment to determine the time of optimal viability. Overview of the treatment groups used in the triploid breeding experiment is shown in Table 1.

TABLE 1
Treatment groups used in triploid breeding.

| Feminized Father | | | |
|---|---|---|---|
| (pollen) | Mother | # of plants | Expected Seed ploidy |
| Tetraploid | Tetraploid | 2 | Tetraploid |
| Tetraploid | Diploid | 6 | Triploid |
| Diploid | Tetraploid | 6 | Triploid |
| Diploid | Diploid | 2 | Diploid |

Once seeds have been generated, they are germinated to determine the percentage of triploids. It is possible that although the somatic cells of a plant are tetraploid, the reproductive cells may still be diploid. If this is the case, all of the seeds will be diploid. Ploidy testing on the seedlings is carried out 6 weeks after germination, as young seedlings sometimes display endopolyploidy, which could confuse results. If enough seed is available, 50 seeds per treatment are germinated and tested. Once triploid seeds are generated, they are grown out and assessed alongside diploid and tetraploid controls to check whether they are morphologically or chemically different from the mother genotypes. Ten plants of each ploidy are monitored for growth rate, speed of flowering onset, yield, chemistry, etc.

In another experiment, four triploid plants are treated to induce male flowers on female plants to check for pollen viability (triploid pollen is theoretically non-viable). Ten female triploid plants are also tested with diploid, triploid, and tetraploid pollen to determine whether mature seeds are generated (two plants of each diploid and tetraploid plants are treated to produce male flowers and pollen), Triploid plants are generally seedless, but “seedless” varieties can produce a small amount of seed. However, significant reductions in pollen viability and/or seed set will confirm that the triploid Cannabis genotypes are improved enough for use in cultivation.

In the first experiment using the Super Nordle® strain, pollen viability and germination assays showed little viability. However, the seed set was good indicating an issue with the assay methods rather than with the male flower induction method. In addition to the seeds collected from female mothers, seeds were gathered from the pollen donor “fathers” (feminized pollen), which yielded a surprising number of seeds, especially in the diploids, Overall, almost 1000 potential triploid seeds were obtained. Results are shown in Table 2.

TABLE 2
Triploid seed generation.

| | | | | | total |
|---|---|---|---|---|---|
| Cross type | Expected | | Average | average | seeds |
| (dad x mom) | Ploidy | n | # seeds/plant | weight seeds (g) | generated |
| tet x tet | Tetraploid | 2 | 36.5 ± 17.5 | 0.01898 ± 0.00004 | 73 |
| tet self | Tetraploid | 4 | 318.5 ± 86.5 | 0.01088 ± 0.00012 | 1274 |
| dip x dip | Diploid | 2 | 722.0 ± 23.3 | 0.01626 ± 0.00144 | 1444 |
| dip self | Diploid | 4 | 1022.2 ± 133.6 | 0.00783 ± 0.00016 | 4089 |
| tet x dip | Triploid | 6 | 88.5 ± 9.1 | 0.00513 ± 0.00011 | 531 |
| dip x tet | Triploid | 6 | 72.5 ± 12.8 | 0.02119 ± 0.00017 | 435 |

It is noted the seed set was poorer in the tetraploid×tetraploid and tetraploid×diploid crosses than in diploid crosses, indicating that viability may be a little poorer in tetraploids, There was no difference in the number of seeds generated from the crosses between diploid and tetraploid “fathers”. However, the seeds produced were significantly larger when diploid “fathers” were used. This might be because the majority of the seed was composed of maternal tissue, and the cells of the maternal tetraploids in these crosses were larger. Crosses between the same ploidy yielded fewer but larger seeds when crossed with a separate plant rather than a self cross, suggesting an effect of hybrid vigour despite the fact that the plants were clones. Next, seeds are germinated from the crosses to confirm ploidy, Triploid plants are then grown and phenotyped to determine whether they are similar or different from their diploid parents. Triploid plants are feminized and challenged with pollen to determine whether pollen viability and seed set are lower in triploids than in diploids.

In the second experiment, the Cannatonic strain is used. Chromosomes are observed and ploidy is assessed using the root tip squash method, flow cytometry, and/or by counting stomata, particularly stomata size and density. Once generated, the triploids are analyzed for growth rate, yield and chemistry and assessed for changes in phenotype or chemical profile compared to diploid control plants.

### Example 2: Triploid Breeding for the Production of Seedless Cannabis

In order to maintain bud quality in growing environments where plants may be exposed to pollen contamination, tetraploid versions of both Palm Tree® CBD and Cannatonic® strains were created and were crossed with their respective diploid counterparts in order to create seedless triploid varieties. The Palm Tree® CBD maternal excess triploids show a 98.4% reduction in seed set when exposed to a pollen challenge, and otherwise show only minor differences in phenotype and chemotype. The few seeds which were obtained from the triploids had a very poor germination rate and produced small, malformed plants. The paternal excess triploids had very poor seed viability, and the plants were generally of poorer quality, with reduced plant health and bud yield.

Experiments were carried out with Palm Tree® CBD and Cannatonic®. To create tetraploids, axillary bud explants were treated with a solution of the mitotic spindle inhibitor oryzalin and grown out in tissue culture (as per the methods described in PCT/CA2019/050332, the entire content of each of which is hereby incorporated by reference). Flow cytometry analysis was used to check the plants for DNA content. Plants which were successfully transformed were rooted and transferred to soil for hardening off. The ploidy was confirmed through visually counting the chromosomes in a root tip squash, as well as re-assessing the ploidy using flow cytometry at several points during the plant's growth (since ploidy reversion is a possibility) Confirmed tetraploids were grown into mother plants and cloned along with regular diploid plants for the next stage of the breeding process.

In order to cross diploids and tetraploids, the plants were induced to create male flowers using a silver thiosulphate spray, Feminized tetraploids were placed with six diploid females and vice versa, so that all seeds produced should be triploid. Two plants of the same ploidy were placed with the feminized plants as a control to compare seed set. Pollen was manually transferred from the males to the females on a weekly basis until harvest. Seeds were harvested from individual plants so that seed generation per plant could be calculated. Based on the results from the PTC interploidy crosses, only diploids were treated as pollen donors in the CAN trials, and tetraploids were used as acceptors. Otherwise, the same procedure was followed.

The resulting seeds were germinated in order to assess viability. Those that germinated were confirmed to be triploid using the flow cytometer. A set of triploid and diploid plants were grown out and phenotyped. Parameters that were assessed included height, stem width, and leaflet width. In addition, both triploids and diploids were exposed to a pollen challenge to check for a reduction in seed set in the triploids, Three of each diploid and triploid plants were treated with STS to become pollen donors, and six acceptor plants of each ploidy were challenged with this pollen, Following the same method as a normal seed run, these plants were pollinated, then harvested to count the number of seeds generated per plant.

Production of Triploids—Interploidy Crosses—Both diploid and tetraploid PTC plants responded well to STS treatment, produced plenty of pollen, and sustained minimal tissue damage. On the other hand, the CAN diploids produced very little pollen during initial crosses. The flowers had to be collected and opened manually in order to disperse pollen onto the acceptors. Seed set was very poor in the CAN crosses as a result, though the genotype could naturally have a low seed set. Seed set in the PTC diploid controls was good, though the tetraploid controls had significantly fewer seeds, possibly indicating that the tetraploid pollen and/or embryos are less viable than the diploids. There was also a low number of seeds from the interploidy crosses, though this was to be expected due to lower compatibility. There was no significant difference in the number of triploid seeds produced between the reciprocal crosses. However, the seeds generated from interploidy crossing were of significantly different sizes than the control diploid seeds. Maternal excess triploid seeds were about 30% larger than diploid seeds, whereas paternal excess triploid seeds were 68% smaller. CAN tetraploids produced significantly fewer seeds than their diploid counterparts, though the weights of the seeds were not substantially different (Table 3).

TABLE 3
Seed production in interploid crosses of two Cannabis genotypes

| | Pollen | Pollen | | |
|---|---|---|---|---|
| Strain | Acceptor | Donor | Seeds per plant | Seed weight (g) |
| PTC | Tetraploid | Tetraploid | 36.5 ± 17.5 a | 0.01898 ± 0.00004 a |
| | Diploid | Diploid | 722.0 ± 23.3 b | 0.01626 ± 0.00144 b |
| | Diploid | Tetraploid | 88.5 ± 9.1 a | 0.00513 ± 0.00011 c |
| | Tetraploid | Diploid | 72.5 ± 12.8 a | 0.02119 ± 0.00017 d |
| CAN | Diploid | Diploid | 22.0 ± 3.0 a | 0.02497 |
| | Tetraploid | Diploid | 1.8 ± 0.7 b | 0.02256 |

Average ± standard error, lowercase italic letters indicate significant differences at p < 0.05.
*Only averages are given for the Cannatonic seed weights since the seeds were grouped for inventory and individual weights were not taken.

Triploid Analysis—Phenotype—The germination rate differed between the maternal and paternal excess triploid progeny of Palm Tree CBD. Although every seed from the maternal excess cross germinated within two weeks, only 1.6% of the paternal excess seeds germinated successfully. As a result, paternal excess triploids were assessed phenotypically, but were not included in the tests for seed set in the triploids. Seeds that did germinate grew normally and did not have any obvious differences from the diploids in the initial vegetative phase. This is a phenomenon which has been observed in other species such as the Brassicas, and is known as “asymmetrical triploid blocking”. Non-viable seeds result from interploidy crosses where the paternal plant is tetraploid due to an overdose of paternally imprinted genes. The gene imbalance interrupts the normal development of the endosperm and deprives the developing embryo of nutrients. Only maternal excess crosses were conducted in Cannatonic® as a result of these findings, and these showed a germination success of 82% two weeks after planting. This is slightly lower than the PTC maternal triploid seeds; however, this strain may have a poorer germination rate in general.

Maternal and paternal triploid PTC clones rooted at approximately the same rate, taking an average of 17.5 and 15.8 days, respectively. Comparatively, the diploid clones took around 20.4 days to root, which was significantly longer than the paternal triploid rooting time. It should be noted, however, that the mother from which the diploid clones were taken was three months older than either of the triploid mothers, which may have contributed to slower rooting. All clones were generally healthy at the end of the rooting period, with some of the diploid clones displaying chlorosis and less growth due to the extended rooting time. All of the triploid clones rooted, but three out of 15 of the diploid clones failed to root within four weeks (an 80% success rate). At 3 weeks after transplant the triploid plants were generally very similar to the diploid plants in appearance. Both sets of triploid plants had significantly wider basal stems, though this was likely another result of being cloned off a younger mother. The triploids also had 16% wider central leaflets, a trait which also appeared in the tetraploid mothers (Table 4). However, in the tetraploids the difference was even more pronounced, indicating that the triploids have an intermediate phenotype. Similarly, the stomata of the triploids showed an intermediate phenotype. The maternal triploids had larger (15%) and less dense (20%) stomata, though not to the same extent as the tetraploids. The paternal triploid stomata were very similar to the diploids (Table 6). The growth characteristics were similar during the flowering stage, with minor differences present between the diploid and triploids (Table 4).

TABLE 4
Plant growth and characteristics of various ploidies of Palm Tree CBD
at 3 weeks (vegetative) and 10 weeks (flower) after transplant

| | | Ploidy | | |
|---|---|---|---|---|
| | | | Maternal | Paternal |
| Characteristic | Stage | Diploid | Triploid | Triploid |
| Height (cm) | Vegetative | 25.4 ± 0.8 a | 24.5 ± 0.5 a | 29.8 ± 0.5 b |
| | Flower | 46.8 ± 1.1 a | 46.3 ± 1.0 a | 51.75 ± 1.4 b |
| Base Stem | Vegetative | 9.3 ± 0.2 a | 13.7 ± 0.4 b | 14.9 ± 0.2 c |
| Width (mm) | Flower | 12.2 ± 0.4 a | 12.5 ± 0.4 a | 16.9 ± 1.1 b |
| Lateral Stem | Vegetative | 141.1 ± 3.8 a | 138.1 ± 2.9 a | 181.6 ± 6.3 b |
| Length (cm) | Flower | 258.2 ± 2.8 a | 264.9 ± 5.6 ab | 280.9 ± 6.3 b |
| Central Leaflet | Vegetative | 26.9 ± 0.3 a | 31.3 ± 1.2 b | 31.4 ± 1.1 b |
| Width (mm) | Flower | 21.5 ± 0.5 a | 23.8 ± 0.5 b | 21.4 ± 0.6 a |

n = 8, average ± standard error. Italicized letters indicate significant differences at p < 0.05.

While all of the plants appeared healthy during the vegetative stage, there were notable differences between the three ploidies by the end of flowering. The diploid plants showed a typical amount of discolouration and leaf senescence associated with the end of the growth cycle, but the paternal triploids showed a much greater degree of tissue necrosis and chlorosis. Conversely, the maternal triploid plants showed even less degradation than the diploids and remained almost entirely green. While the maternal triploids had a modest but significant increase in wet yield compared to the diploid controls (13.5%), the dry yield weights were nearly identical. On the other hand, the unhealthy-looking paternal triploids had a significantly lower dry bud weight than the control (Table 5).

TABLE 5
Harvest weights of Palm Tree CBD triploids.

| Ploidy | Whole Plant (g) | Bud (g) | Proportion (%) | Dry Bud (g) |
|---|---|---|---|---|
| Diploid | 366.8 ± 7.8 a | 287.1 ± 5.8 a | 78.3 ± 0.4 a | 81.5 ± 1.6 a |
| Maternal | 424.8 ± 6.1 b | 325.6 ± 6.2 b | 76.6 ± 1.0 a | 81.6 ± 2.7 a |
| Triploid | | | | |
| Paternal | 426.5 ± 11.3 b | 293.5 ± 8.6 a | 68.8 ± 1.0 b | 65.8 ± 2.3 b |
| Triploid | | | | |

n = 8, average ± standard error. Italicized letters indicate significant differences at p < 0.05. Proportion indicates the percent of the total plant weight which was bud.

TABLE 6
Stomata characteristics of Palm Tree CBD triploids during
the vegetative phase

| | Stomata Density | Stomata | Guard Cell |
|---|---|---|---|
| Ploidy | (#/mm²) | Length (μm) | Width (μm) |
| Diploid | 579.2 ± 30.3 a | 16.57 ± 0.28 a | 5.35 ± 0.1 a |
| Maternal Triploid | 464.6 ± 21.0 b | 19.45 ± 0.45 b | 6.23 ± 0.20 b |
| Paternal Triploid | 458.3 ± 29.4 b | 17.51 ± 0.41 a | 5.83 ± 0.18 ab |

n = 8, 24, and 24, respectively, average ± standard error. Italicized letters indicate significant differences at p < 0.05.

Triploid Analysis—Chemotype: While the maternal triploids had a very similar cannabinoid profile to the diploids (with minor reductions in the main cannabinoids), the paternal triploids had a completely unique cannabinoid profile. Unexpectedly, the paternal triploids showed significant reductions in nearly all cannabinoids present, including almost total absence of CBDA. On the other hand, the paternal triploids had a large increase in THCA, 43% over the diploids (Table 7). The maternal triploids had higher variability between individual samples, with several individuals having higher cannabinoid content than the diploids, but ultimately averaging out to a slightly lower cannabinoid content.

TABLE 7
Alterations in cannabinoid concentrations in three ploidies of Palm Tree ® CBD.

| Cannabinoid Content | Ploidy | | |
|---|---|---|---|
| (% w/w) | Diploid | Maternal Triploid | Paternal Triploid |
| Cannabicyclol | 0.0 ± 0.0 a | 0.0 ± 0.0 a | 0.0 ± 0.0 a |
| Cannabidivarin | 0.0 ± 0.0 a | 0.0 ± 0.0 a | 0.0 ± 0.0 a |
| Cannabidivarinic acid | 0.047 ± 0.001 a | 0.040 ± 0.004 b | 0.0 ± 0.0 c |
| Cannabichromenic Acid | 0.512 ± 0.006 a | 0.468 ± 0.022 a | 0.101 ± 0.014 b |
| Tetrahydrocannabivarin | 0.188 ± 0.004 a | 0.160 ± 0.009 b | 0.119 ± 0.003 c |
| Cannabinol | 0.0 ± 0.0 a | 0.0 ± 0.0 a | 0.0 ± 0.0 a |
| Cannabichromene | 0.0 ± 0.0 a | 0.0 ± 0.0 a | 0.0 ± 0.0 a |
| Δ⁸-tetrahydrocannabinol | 0.0 ± 0.0 a | 0.0 ± 0.0 a | 0.004 ± 0.003 a |
| Cannabigerol | 0.0 ± 0.0 a | 0.0 ± 0.0 a | 0.0 ± 0.0 a |
| Cannabigerolic acid | 0.213 ± 0.013 a | 0.257 ± 0.028 a | 0.090 ± 0.003 b |
| Cannabidiol | 0.132 ± 0.005 a | 0.185 ± 0.012 b | 0.0 ± 0.0 c |
| Cannabidiolic Acid | 10.182 ± 0.119 a | 9.589 ± 0.683 a | 0.058 ± 0.001 b |
| Δ⁹-tetrahydrocannabinol | 0.246 ± 0.006 a | 0.310 ± 0.046 a | 0.434 ± 0.008 b |
| Δ⁹-tetrahydrocannabinolic acid | 7.768 ± 0.088 a | 6.744 ± 0.871 a | 11.135 ± 0.252 b |
| Potential CBD | 9.27 ± 0.110 a | 8.772 ± 0.622 a | 0.051 ± 0.001 b |
| Potential THC | 7.215 ± 0.079 a | 6.351 ± 0.827 a | 10.339 ± 0.234 b |
| Total Cannabinoids | 18.911 ± 0.257 a | 17.085 ± 0.453 b | 11.836 ± 0.269 c |

(n. = 8, average ± standard error). Lowercase letters indicate significant differences at p < 0.05.

The maternal triploid plants showed very few changes in the terpene profile compared to the control, only the concentration of b-caryophyllene was decreased (approximately 45%). However, the concentration of this terpene was not above the 0.5 mg/g concentration threshold for biological activity in either the diploid or triploid, so the physiological effects would not change. The paternal triploid plants had a 30% overall reduction in terpenes, with significant decreases in several major terpenes compared to the diploids (Table 8). It is likely that the paternal triploids have a reduced smell compared to the diploids, and may also have altered taste or biological activity.

TABLE 8
Alterations in terpene concentrations in three ploidies of Palm Tree ® CBD.

| Terpene Content | Terpene | Ploidy | | |
|---|---|---|---|---|
| (% w/w) | Class | Diploid | Maternal Triploid | Paternal Triploid |
| α-Pinene | monoterpene | 0.096 ± 0.005 a | 0.087 ± 0.005 a | 0.055 ± 0.005 b |
| Camphene | monoterpene | 0.0 ± 0.0 a | 0.0 ± 0.0 a | 0.0 ± 0.0 a |
| β-Pinene | monoterpene | 0.045 ± 0.002 a | 0.043 ± 0.002 ab | 0.034 ± 0.003 b |
| Myrcene | monoterpene | 0.327 ± 0.031 a | 0.335 ± 0.029 a | 0.224 ± 0.022 b |
| delta-3-Carene | monoterpene | 0.0 ± 0.0 a | 0.0 ± 0.0 a | 0.0 ± 0.0 a |
| α-Terpinene | monoterpene | 0.0 ± 0.0 a | 0.0 ± 0.0 a | 0.0 ± 0.0 a |
| p-Cymene | monoterpene | 0.0 ± 0.0 a | 0.0 ± 0.0 a | 0.0 ± 0.0 a |
| Limonene | monoterpene | 0.032 ± 0.002 a | 0.034 ± 0.003 a | 0.028 ± 0.002 a |
| Eucalyptol | monoterpene | 0.0 ± 0.0 a | 0.0 ± 0.0 a | 0.0 ± 0.0 a |
| Ocimene | monoterpene | 0.002 ± 0.001 ab | 0.004 ± 0.001 a | 0.0 ± 0.0 b |
| g-Terpinene | monoterpene | 0.0 ± 0.0 a | 0.0 ± 0.0 a | 0.0 ± 0.0 a |
| Terpinolene | monoterpene | 0.0 ± 0.0 a | 0.0 ± 0.0 a | 0.0 ± 0.0 a |
| Linalool | monoterpene | 0.023 ± 0.001 a | 0.021 ± 0.001 a | 0.013 ± 0.001 b |
| Isopulegol | monoterpene | 0.0 ± 0.0 a | 0.0 ± 0.0 a | 0.0 ± 0.0 a |
| α-Terpineol | monoterpene | 0.0 ± 0.0 a | 0.0 ± 0.0 a | 0.0 ± 0.0 a |
| g-Terpineol | monoterpene | 0.049 ± 0.002 a | 0.049 ± 0.006 a | 0.037 ± 0.002 a |
| Geraniol | monoterpene | 0.016 ± 0.001 a | 0.013 ± 0.003 a | 0.003 ± 0.002 b |
| β-Caryophyllene | sesquiterpene | 0.035 ± 0.002 a | 0.019 ± 0.003 b | 0.044 ± 0.003 a |
| αa-Humulene | sesquiterpene | 0.0 ± 0.0 a | 0.0 ± 0.0 a | 0.0 ± 0.0 a |
| Guaiol | sesquiterpene | 0.001 ± 0.000 a | 0.001 ± 0.000 a | 0.0 ± 0.0 a |

| | | | |
|---|---|---|---|
| Total Terpenes | 0.628 ± 0.044 a | 0.606 ± 0.044 a | 0.438 ± 0.041 b |

(n = 8, average ± standard error). Lowercase letters indicate significant differences at p < 0.05.

Any element of any embodiment may be used in any embodiment. Although the technology has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention. Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

All references cited in this specification, and their references, are incorporated by reference herein in their entirety where appropriate for teachings of additional or alternative details, features, and/or technical background.

### REFERENCES

Abdoli, M., Moieni, A., and Bach, H. N. (2013). Morphological, physiological, cytological and phytochemical studies in diploid and colchicine-induced tetraploid plants of Echinacea purpurea (L.). Acta physiologiae plantarum 35, 2075-2083. doi: 10.1007/s11738-013-1242-9
Adams, K. L., and Wendel, J. F. (2005). Polyploidy and genome evolution in plants. Current opinion in plant biology 8, 135-141, doi.: 10.1016/j.pbi.2005.01.001
Andre, C. M., Hausman, J. F., and Guerriero, G. (2016). Cannabis sativa: the plant of the thousand and one molecules. Frontiers in plant science 7, 19. doi: 10.3389/fpls.2016.00019
Ascough, G. D., Van Staden, J., and Erwin, J. E. (2008). Effectiveness of colchicine and oryzalin at inducing polyploidy in Watsonia lepida NE Brown. HortScience 43, 2248-2251
Bagheri, M., and Mansouri, H. (2015), Effect of induced polyploidy on some biochemical parameters in Cannabis sativa L. Applied biochemistry and biotechnology 175, 2366-2375. doi: 10.1007/s12010-014-1435-8
Beatson, R. A., Ansell, K. A., and Graham, L. T. (2003). Breeding, development, and characteristics of the hop (Humulus lupulus) cultivar ‘Nelson Sauvin’. New Zealand Journal of Crop and Horticultural Science 31, 303-309.
Beatson, R. A., Ferguson, A. R., Weir, I. E., Graham, L. T., Ancell, K. A. and Ding, H. (2003) Flow cytometric identification of sexually derived polyploids in hop (Humulus lupulus L.) and their use in hop breeding. Euphytica 134(2), 189-194.
Berkov, S., and Philipov, S. (2002). Alkaloid production in diploid and autotetraploid plants of Datura stramonium. Pharm. Biol. 40, 617-621. doi: 10.1076/phbi.40.8.617.14650
Blakeslee, A. F., and Avery, A. G. (1937). Methods of inducing doubling of chromosomes in plants: by treatment with colchicine. journal of Heredity 28, 393-411. doi: 10.1093/oxfordjournals.jhered.a104294
Brenneisen, R. (2007). Chemistry and analysis of phytocannabinoids and other Cannabis constituents. In: Elsohly M (ed.). Marijuana and the Cannabinoids. Humana Press: Totowa, N.Y., pp. 17-49
Chaohua, C., Gonggu, Z., Lining, Z., Chunsheng, G., Qing, T., Jianhua, C., et al. (2016). A rapid shoot regeneration protocol from the cotyledons of hemp (Cannabis sativa L.). Industrial Crops and Products 83, 61-65. doi: 10.1016/j.indcrop.2015.12.035
Chen, C. H., & Goeden-Kallemeyn, Y. C. (1979). In vitro induction of tetraploid plants from colchicine-treated diploid daylily callus. Euphytica 28, 705-709. doi: 10.1007/BF00038937
Clarke, R. C. (1981), “Chapter 3: The Genetics and Breeding of Cannabis” in Marijuana botany: An advanced study: The propagation and breeding of distinctive cannabis. (Berkeley, Calif.: Ronin publishing), 27-59. Retrieved from https://www.thevespiary.org/library/Files_Uploaded_by_Users/llamabox/Clarke,%20Robert %20C %20%20Marijuana %20Botany %20An %20Advanced %20Study.pdf on 29/08/2018
Comai, L. (2005). The advantages and disadvantages of being polyploid. Nature reviews genetics 6, 836. doi:10.1038/nrg1711
Contreras, R. N., and Meneghelli, L. (2016). In vitro chromosome doubling of Prunus laurocerasus ‘Otto Luyken’ and ‘Schipkaensis’. HortScience 51, 1463-1466. doi: 10.21273/HORTSCI11329-16
Corroon, J., and Phillips, J. A. (2018). A cross-sectional study of cannabidiol users. Cannabis and Cannabinoid Res. 3, 152-161. doi: 10.1089/can.2018.0006
De Meijer, E. P. M., Bagatta, M., Carboni, A., Crucitti, P., Moliterni, V. M. C., Ranalli, P., et al. (2003). The inheritance of chemical phenotype in Cannabis sativa L. Genetics 163, 335-346. doi: 10.1007/s10681-005-1164-8
De Moraes Pultrini, A., Galindo, L. A., and Costa, M. (2006). Effects of the essential oil from Citrus aurantium L. in experimental anxiety models in mice. Life Sciences 78, 1720-1725. doi: 10.1016/j.lfs.2005.08.004
Dermen, H. (1940). Colchicine polyploidy and technique. The botanical review 6, 599-635. doi: 10.1007/BF02919557
Dhooghe, E., Grunewald, W., Letts, L., and Van Labeke, M. C. (2009). In vitro polyploidisation of Helleborus species. Euphytica 165, 89-95. dot: 10.1007/s10681-008-9763-9
Divashuk, M. G., Alexandrov, O. S., Razumova, O. V., Kirov, I. V., and Karlov, G. I. (2014). Molecular cytogenetic characterization of the dioecious Cannabis sativa with an XY chromosome sex determination system. PLUS One 9, e85118. doi: 10.1371/journal.pone.0085118
Doležel, J., Binarová, P., and Lucretti, S. (1989). Analysis of nuclear DNA content in plant cells by flow cytometry. Biologia plantarum 31, 113-120. doi: 10.1007/BF02907241
Doležel, J., Sgorbati, S., and Lucretti, S. (1992). Comparison of three DNA fluorochromes for flow cytometric estimation of nuclear DNA content in plants. Physiol. Plant. 85, 625-631. doi: 10.1111/j.1399-3054.1992.tb04764.x
ElSohly, M. A. (2016) The botany of Cannabis sativa L, in The Analytical Chemistry of Cannabis (pp. 1-26). Amsterdam, Netherlands, Elsevier Inc. doi: 10.1016/B978-0-12-804646-3.00001-1
Fernandes, E. S., Passos, G. F., Medeiros, R., da Cunha, F. M., Ferreira, J., Campos, M. M. et al. (2007). Anti-inflammatory effects of compounds alpha-humulene and (−)-trans-caryophyllene isolated from the essential oil of Cordia verbenacea. European journal of pharmacology 569, 228-236. doi: 10, 1016/j.ejphar.2007.04.059
Flores-Sanchez, I., and Verpoorte, R. (2008). Secondary metabolism in cannabis. Phytochem. Rev. 7, 615-639. doi: 10.1007/s11101-008-9094-4
Grant, B, W., and Vatnick, I, (2004). Environmental correlates of leaf stomata density. Teaching Issues and Experiments in Ecology. (Ecological Society of America: Washington, D. C.). Retrieved from https://tiee.esa.org/vol/v1/experiments/stomata/pdf/stomata.pdf on 23/10/2018.
Happyana, N. (2014) Metabolomics, Proteomics, and Transcriptomics of Cannabis sativa L. Trichomes. [dissertation/doctoral thesis][Germany] Technical University of Dortmund
Health Canada (2013). Information for Health Care Professionals: Cannabis (marihuana, marijuana) and the cannabinoids. Obtained from https://www.canada.ca/en/health-canada/services/drugs-medication/
Kamatou, G. P., and Viljoen, A, M, (2010). A review of the application and pharmacological properties of α-Bisabolol and α-Bisabolol-rich oils. Journal of the American oil chemists' society 87, 1-7. doi: 10.1007/s11746-009-1483-3
Koutoulis, A., Roy, A. T., Price, A., Sherriff, L. and Leggett, G. (2005). DNA ploidy level of colchicine-treated hops (Humulus lupulus L.). Scientia Horticulturae 105, 263-268.
Lam. H. K., Harbard, J. L., & Koutoulis, A. (2014). Tetraploid Induction of Acacia crassicarpa using colchicine and oryzalin. Journal of Tropical Forest Science, 347-354,
Lata, H., Chandra, S., Techen, N., Khan, I. A., and ElSohly, M. A. (2016). In vitro mass propagation of Cannabis sativa L.: A protocol refinement using novel aromatic cytokinin meta-topolin and the assessment of eco-physiological, biochemical and genetic fidelity of micropropagated plants. Journal of Applied Research on Medicinal and Aromatic Plants 3, 18-26. doi: 10.1016/j.jarmap.2015, 12.001
Lata, H., Chandra, S., Khan, I., and ElSohly, M. A. (2009). Thidiazuron-induced high-frequency direct shoot organogenesis of Cannabis sativa L. In Vitro Cellular & Developmental Biology-Plant 45, 12-19. doi.: 10.1007/s11627-008-9167-5
Laverty, K. U., Stout, J. M., Sullivan, M. J., Shah, H., Gill, N., Holbrook, L., et al. (2018). A physical and genetic map of Cannabis sativa identifies extensive rearrangement at the THC/CBD acid synthase locus Genome Res. 29, 1-11. doi: 10.1101/gr.242594.118
Majdi, M., Karimzadeh, G., Malboohi, M. A., Omidhaigi, R. and Mirzaghaderi, G. (2010). Induction of tetraploidy to feverfew (Tanacetum parthenium Schulz-Bip.): Morphological, physiological, cytological, and phytochemical changes. HortScience 45, 16-21
Mansouri, H., and Bagheri, M. (2017). “The induction of polyploidy and its effect on Cannabis sativa L.,” in Cannabis sativa L.—Botany and Biotechnology, eds. S. Chandra, H. Lata & M. A. Elsohly. (New York, N.Y.: Springer International Publishing).
Marks, M. D., Tian, L., Wenger, J. P., Omburo, S. N., Soto-Fuentes, W., He, J., et al. (2009). Identification of candidate genes affecting Δ⁹-tetrahydrocannabinol biosynthesis in Cannabis sativa J. Exp. Bot. 60, 3715-3726. doi: 10.1093/jxb/erp210
Martin, S. L., Sauder, C. A., James, T., Cheung, K. W., Razeq, F. M., Kron, P., et al. (2015). Sexual hybridization between Capsella bursa-pastoris (L.) Medik (2) and Camelina sativa (L.) Crantz (♂) (Brassicaceae). Plant Breed. 134, 212-220. doi: 10.1111/pbr.12245
Maurya, A., Singh, M., Duhey, V., Srivastava, S., Luqman, S., and Bawankule, D. (2014). α-(−)-bisabolol reduces pro-inflammatory cytokine production and ameliorates skin inflammation. Current pharmaceutical biotechnology 15, 173-181
Mechoulam, R., Parker, L. A., and Gallily, R. (2002). Cannabidiol: an overview of some pharmacological aspects. The Journal of Clinical Pharmacology 42, 11S-19S. doi: 10.1002/j.1552-4604.2002. tb05998.x
Meru, G. M. (2012). “Polypoidy and its implications in plant breeding,” in Plant breeding in the 21st century, eds. C. Mcgregor & C. Brummer. (Athens, Ga.: University of Georgia).
Mishra, B. K., Pathak, S., Sharma, A., Trivedi, P. K., and Shukla, S. (2010). Modulated gene expression in newly synthesized auto-tetraploid of Papaver somniferum L. S. Afr. J. Bot. 76, 447-452. doi: 10.1016/j. sajb.2010.02.090
Morejohn, L. C., Bureau, T. E., Molé-Bajer, J., Bajer, A. S., and Fosker, D. E. (1987). Oryzalin, a dinitroaniline herbicide, binds to plant tubulin and inhibits microtubule polymerization in vitro. Planta 172, 252-264.
Parsons, J. L., Martin, S. L., James, T., Golenia, G., Boudko, E. A., and Hepworth, S. R. (2019). Polyploidization for the Genetic Improvement of Cannabis sativa. Front. Plant Sci. 10: 476. doi: 10.3389/fpls.0.2019.00476
Passos, G. F., Fernandes, E. S., da Cunha, F. M., Ferreira, Pianowski, L. F., Campos, M. M., and Calixto, J. B. (2007). Anti-inflammatory and anti-allergic properties of the essential oil and active compounds from Cordia verbenacea, Journal of ethnopharmacology 110, 323-333. doi: 10.1016/j.jep.2006.09.032
Petersen, K. K., Hagberg, P., and Kristiansen, K. (2003). Colchicine and oryzalin mediated chromosome doubling in different genotypes of Miscanthus sinensis. Plant Cell, Tissue and Organ Culture 73, 137-146. doi: 10.1023/A:1022854303371
Ram, H. Y. M. and Jaiswal, V. S. (1972). Induction of Male Flowers on Female Plants of Cannabis sativa by Gibberellins and Its Inhibition by Abscisic Acid. Planta 105, No. 3, 263-266.
Ram, H. Y. M. and Sett, R. (1979). Sex reversal in the female plants of Cannabis sativa by cobalt ion. Proc. Indian Acad. Sci. 88B, Part II, No. 4, 303-308.
Ram, H. Y. M. and Sett, R. (1982). Induction of Fertile Male Flowers in Genetically Female Cannabis sativa Plants by Silver Nitrate and Silver Thiosulphate Anionic Complex. Theor. Appl. Genet. 62, 369-375.
Rêgo, M. D., Rêgo, E. R., Bruckner, C. H., Finger, F. L., and Otoni, W. C. (2011). In vitro induction of autotetraploids from diploid yellow passion fruit mediated by colchicine and oryzalin. Plant Cell, Tissue and Organ Culture 107, 451-459. doi: 10.1007/s11240-011-9995-6
Roy, A., Leggett, G., & Koutoulis, A. (2001). In vitro tetraploid induction and generation of tetraploids from mixoploids in hop (Humulus lupulus L.), Plant cell reports 20, 489-495, doi: 10.1007/s002990100364
Russo, E. B, (2011), Taming THC: potential cannabis synergy and phytocannahinoid-terpenoid entourage effects. British journal of pharmacology 163, 1344-1364. doi:10, 1111/j.1476-5381.2011.01238.x
Sakhanokho, H. F., Rajasekaran, K., Kelley, R. Y., and Islam-Faridi, N. (2009). Induced polyploidy in diploid ornamental ginger (Hedychium muluense R M Smith) using coichicine and oryzalin. HortScience 44, 1809-1814.
Sarath, G. and Ram, H. Y. M. (1978). Comparative effect of silver ion and gibberellic acid on the induction of male flowers on female Cannabis plants. Experientia 35/3, 333-334.
Sattler, M. C., Carvalho, C. R., and Clarindo, W. R. (2016). The polyploidy and its key role in plant breeding. Planta 243, 281-296. doi: 10.1007/s00425-015-2450-x
Smith, I. MV., Kron, P., and Martin, S. L. (2018). flowPioidy: An R package for genome size and ploidy assessment of flow cytometry data. Applications in plant sciences 6. doi: 10.1002/aps3.1164
Stanys, V., Weckman, A., Staniene. G., and Duchovskis, P. (2006). In vitro induction of polyploidy in Japanese quince (Chaenomeles japonica). Plant Cell, Tissue and Organ Culture 84, 263-268. doi: 10.1007/s11240-005-9029-3
Talebi, S. F., Saharkhiz, M. J., Kermani, M. J., Sharafi, Y., and Raouf Fard, F. (2017). Effect of different antimitotic agents on polyploid induction of anise hyssop (Agastache foeniculum L.). Caryologia 70, 184-193. doi: 10.1080/00087114.2017.1318502
Tavan, M., Mirjalili, M. H., and Karimzadeh, G. (2015), in vitro polyploidy induction: changes in morphological, anatomical and phytochemical characteristics of Thymus persicus (Lamiaceae). Plant Cell, Tissue and Organ Culture 122, 573-583. doi: 10.1007/s11240-015-0789-0
Thomas, B. F., and Elsohly, M. A. (2016). “The botany of Cannabis sativa L.,” in The Analytical Chemistry of Cannabis. (Amsterdam, Netherlands: Elsevier Inc.), 1-26.
Trojak-Goluch, A., and Skomra, U. (2013). Artificially induced polyploidization in Humulus lupulus L. and its effect on morphological and chemical traits. Breeding science 63, 393-399. doi: 10.1270/jsbbs.63.393
Tsuchiya, T., and Nakamura., C. (1979). Acetocarmine squash method for observing sugar beet chromosomes, Euphytica 28, 249-256. doi: 10.1007/BF00056582
Van Bakel, H., Stout, J. M., Cote, A. G., Tallon, C. M., Sharpe, A. G., Hughes, T. R., et al. (2011). The draft genome and transcriptome of Cannabis sativa. Genome Biol. 12, R102. doi: 10.1186/gb-2011-12-10-r102
Viehmannová, I., Cusimamani, E. F. Bechyne, M., Vyvadilová, M., & Greplová, M. (2009). In vitro induction of polyploidy in yacon (Smallanthus sonchifolius). Plant Cell, Tissue and Organ Culture 97, 21-25. doi: 10.1007/s11240-008-9494-6
Wallaart, T. E., Pras, N., and Quax, W. (1999). Seasonal variation of artemis and its biosynthetic precursors in tetraploid Artemisia annua plants compared to the diploid wild-type. Planta Medica 65, 723-728. doi: 10.1055/s-1999-14094
Wilkinson, J. D., Whalley, B. J., Baker, D., Pryce, G., Constanti, A., Gibbons, S., and Williamson, E. M. (2003). Medicinal cannabis: is Δ9-tetrahydrocannabinol necessary for all its effects? Journal of Pharmacy and Pharmacology 55, 1687-1694. doi: 10.1211/0022357022304

1. A triploid Cannabis plant, comprising one or more of:
i) a Cannabidivarinic acid (CBDVA) content of between about 0.05% and about 0.1% w/w;
ii) a Cannabichromenic acid (CBCA) content of between about 0.05% and about 1.0% w/w;
iii) a Tetrahydrocannabivarin (THCV) content of between about 0.05% and about 0.3% w/w;
iv) a Δ⁸-tetrahydrocannabinol (Δ⁸-THC) content of between about 0.001% and about 0.01% w/w;
v) a Cannabigerolic acid (CBGA) content of between about 0.01% and about 0.5% w/w;
vi) a Cannabidiol (CBD) content of between about 0% and about 0.5% w/w;
vii) a Cannabidiolic acid (CBDA) content of between about 0% and about 20% w/w;
viii) a Δ⁹-tetrahydrocannabinol (THC) content of between about 0.1% and about 1.0% w/w; and
ix) a Δ⁹-tetrahydrocannabinolic acid (THCA) content of between about 0.1% and about 30% w/w.

2. The triploid Cannabis plant of claim 1 comprising a Δ⁹-tetrahydrocannabinolic acid (THCA) content of between about 0.1% and about 30% w/w.

3. The triploid Cannabis plant of claim 1, comprising a Δ⁹-tetrahydrocannabinolic acid (THCA) content of between about 0.1% and about 30% w/w and a Cannabidiolic acid (CBDA) content of between about 0% and about 20% w/w.

4. The triploid Cannabis plant of claim 1, comprising a Δ⁹-tetrahydrocannabinolic acid (THCA) content of between about 0.1% and about 30% w/w; a Cannabidiolic acid (CBDA) content of between about 0% and about 20% w/w; and a Cannabigerolic acid (CBGA) content of between about 0.01% and about 0.5% w/w.

5. The triploid Cannabis plant of claim 1, comprising a Δ⁹-tetrahydrocannabinolic acid (THCA) content of between about 0.1% and about 30% w/w; a Cannabidiolic acid (CBDA) content of between about 0% and about 20% w/w; and a Cannabichromenic acid (CBCA) content of between about 0.05% and about 1.0% w/w.

6. The triploid Cannabis plant of claim 1, comprising a Δ⁹-tetrahydrocannabinolic acid (THCA) content of between about 0.1% and about 30% w/w; a Cannabidiolic acid (CBDA) content of between about 0% and about 20% w/w; a Cannabigerolic acid (CBGA) content of between about 0.01% and about 0.5% w/w; and a Cannabichromenic acid (CBCA) content of between about 0.05% and about 1.0% w/w.

7. The triploid Cannabis plant of claim 2, further comprising a Δ⁸-tetrahydrocannabinol (Δ⁸-THC) content of between about 0.001% and about 0.01% w/w.

8. The triploid Cannabis plant of claim 2, further comprising a Tetrahydrocannabivarin (THCV) content of between about 0.05% and about 0.3% w/w.

9. The triploid Cannabis plant of claim 1, further comprising a total terpene content of between about 0.1% and about 2.0% w/w.

10. The triploid Cannabis plant of claim 1, further comprising one or more of: i) a α-Pinene content of between about 0.04% and about 0.1% w/w; ii) a Myrcene content of between about 0.1% and about 0.5% w/w; and iii) a β-Caryophyllene content of between about 0.01% and about 0.05% w/w.

11. The triploid Cannabis plant of claim 1, wherein the triploid Cannabis plant is a Cannabis sativa plant.

12. The triploid Cannabis plant of claim 1, wherein the triploid Cannabis plant is a hemp plant.

13.-65. (canceled)

66. The triploid Cannabis plant of claim 1, comprising a Δ⁹-tetrahydrocannabinolic acid (THCA) content of between about 0.1% and about 20% w/w.

67. The triploid Cannabis plant of claim 1, comprising a Δ⁹-tetrahydrocannabinolic acid (THCA) content of between about 0.1% and about 10% w/w.

68. The triploid Cannabis plant of claim 1, comprising a Cannabidiolic acid (CBDA) content of between about 0% and about 20% w/w.

69. The triploid Cannabis plant of claim 1, comprising a Cannabidiolic acid (CBDA) content of between about 0% and about 15% w/w.

70. The triploid Cannabis plant of claim 1, comprising a Cannabidiolic acid (CBDA) content of between about 0% and about 10% w/w.
